(19) United States
(12) Patent Application Publication
(10) Pub. No.: US 2026/0345209 A1
(43) Pub. Date: Nov. 5, 2026

(54) SPINAL FIXATION

(71) Applicant: Providence Medical Technology, Inc., Pleasanton, CA (US)

(72) Inventors: Edward Liou, Danville, CA (US); Mathew Tivey, Livermore, CA (US); Bon Champ, Campbell, CA (US)

(21) Appl. No.: 19/119,934

(22) PCT Filed: Oct. 11, 2023
(86) PCT No.: PCT/US2023/034882

§ 371 Date: Apr. 10, 2025

Related U.S. Application Data
(60) Provisional application No. 63/415,094, filed on Oct. 11, 2022.

(51) Int. Cl. A61B 17/70; A61B 17/17

(52) U.S. Cl. CPC A61B 17/7064; A61B 17/1757; A61B 17/7001

(57) ABSTRACT

A spinal fixation device includes a cage member configured for insertion into a facet joint and an articulating member attached or coupled to the cage member. The device includes a ball joint, a pivot joint, or both configured to allow the position, orientation and/or angle of the articulating member to be adjusted after placement of the device at a facet joint. An articulating member can receive and support a connecting rod and can include a screw member or portion insertable into one or more receiving holes defined by a cage member. A spinal fixation system includes a delivery instrument configured to deliver the spinal fixation device to a target site, which may include a facet joint and one or more articular processes adjacent thereto. The system may include a drill bit and associated methods of use may involve creating a pilot hole through an articular process for subsequent insertion of a screw.

### CROSS-REFERENCE TO RELATED APPLICATION

This application claims the benefit of U.S. Provisional Application No. 63/415,094, filed Oct. 11, 2022, the disclosure of which is hereby incorporated by reference in its entirety.

### TECHNICAL FIELD

This disclosure is directed to medical devices, systems and methods. More specifically, this disclosure is directed to devices, systems and methods related to spinal fixation procedures.

### BACKGROUND

Chronic back problems are one of the most common causes of pain and disability in the United States and other developed countries. According to at least one estimate, spinal fusion procedures, in which two adjacent vertebrae are fused together using plates, screws and other implants, are the most commonly performed surgical procedures in the United States. Spinal fusion is often performed in an attempt to increase space between the two adjacent vertebrae being operated on (known as spinal distraction) and to thus prevent impingement of the spinal cord or nerve roots branching from the spinal cord and passing through openings in the vertebral column. Unfortunately, most techniques and devices used for performing spinal fusion are invasive, high-risk, and require difficult recovery and rehabilitation.

Accordingly, a need exists for new, less invasive devices and methods for fixing, and stabilizing one or more vertebrae of the spine to promote fusion.

### SUMMARY

The present disclosure describes devices, systems and associated methods for spinal procedures. In accordance with embodiments of the present disclosure, a device for spinal fixation includes a cage member defining a body configured for insertion into a facet joint, an articulating member configured to receive a connecting rod and coupled to a proximal end of the cage member via at least one pivot joint.

In some examples, the articulating member may define a connecting rod receiving slot defining a proximal open end and a concave resting surface opposite the proximal open end and complementary to the connecting rod. In some examples, the pivot joint may include a pivot pin and a pivot member, the pivot member defining an inner lumen configured to receive the pivot pin. In some examples, the proximal end of the cage member may define opposing shoulder portions each defining through-holes configured to receive the pivot pin and align concentrically with the inner lumen of the pivot member. In some examples, the device further includes a ball joint coupled to the at least one pivot joint and positioned at least partially within a cavity defined by the articulating member. The ball joint may include a ball member coupled to a shaft member, the shaft member extending from the pivot member. In some examples, the shaft member may be arranged substantially perpendicular to the inner lumen of the pivot member. In some examples, an exterior surface of the shaft member may be substantially smooth. In some examples, an exterior surface of the shaft member may be threaded. In some examples, the articulating member may be configured to pivot about and/or slide along the pivot joint. In some examples, the body of the cage member may include protrusions configured to engage the facet joint. In some examples, a ball joint is coupled to the at least one pivot joint and positioned at least partially within a cavity defined by the articulating member such that the articulating member is configured to rotate about the ball joint. In some examples, the device may also include a cap coupled to the ball joint within the cavity defined by the articulating member and defining concave surfaces complementary to the connecting rod. In some examples, the at least one pivot joint includes an articulating joint and a pivot joint.

In accordance with some embodiments of the present disclosure, a device for spinal fixation includes a cage member defining a body comprising two arm members configured to engage an articular process therebetween. The device may also include an articulating member configured to receive a connecting rod and defining an elongate screw portion, the elongate screw portion defining a threaded exterior surface configured to extend through and engage complementary holes defined by the arm members. The device may also include a ball joint coupled to a proximal end of the elongate screw member and positioned at least partially within a cavity defined by the articulating member.

In some examples, one of the two arm members may be configured for insertion into a facet joint. In some examples, each of the complementary holes defined by the arm members may define a threaded interior surface complementary to the threaded exterior surface of the elongate screw portion. In some examples, the device may also include a nylon insert configured to be inserted into one of the holes defined by the arm members. In some examples, the device may also include a lock shim insert configured to be inserted into a complementary side slot defined by one of the two arm members. In some examples, the holes defined by the arm members may be configured to receive a drill bit. In some examples, the device may also include a coupling mechanism for coupling the device with a delivery instrument. In some examples, the coupling mechanism may also include a threaded receiving hole configured to receive a threaded screw portion extending from a distal end of the delivery instrument. In some examples, the articulating member may be configured to rotate about the ball joint. In some examples, the articulating member may define a connecting rod receiving slot defining a proximal open end and a concave resting surface opposite the proximal open end and complementary to the connecting rod.

In accordance with some embodiments of the present disclosure, a device for spinal fixation may include a cage member comprising a body defining a receiving hole and configured to engage an articular process. The device may also include an articulating member configured to receive a connecting rod and defining an elongate screw portion, the elongate screw portion defining a threaded exterior surface configured to engage the receiving hole defined by the body of the cage member such that a distal end of the elongate screw portion is seated within the receiving hole. The device may also include a ball joint coupled to a proximal end of the elongate screw member and positioned at least partially within a cavity defined by the articulating member.

In some examples, embodiments of a spinal fixation system disclosed herein may also include a delivery instrument having an elongate body configured for coupling to at least a proximal portion of the spinal fixation device.

In some examples, embodiments of a spinal fixation system disclosed herein may also include a delivery instrument comprising an elongate body and including an adapter member configured to couple with the cage member and defining a through-hole configured to receive and guide a drill bit through an articular process and the one or more holes defined by the cage member.

In some examples, the spinal fixation system may also include a connecting rod configured to couple with the articulating member of the spinal fixation device.

### BRIEF DESCRIPTION OF THE DRAWINGS

FIG. 1A is an exploded view of a spinal fixation device in accordance with embodiments of the present disclosure.

FIG. 1B is a perspective view of the spinal fixation device of FIG. 1A and a corresponding delivery instrument in accordance with embodiments of the present disclosure.

FIG. 1C is a perspective view of the spinal fixation device coupled with the delivery instrument in accordance with embodiments of the present disclosure.

FIG. 1D is a side view of the spinal fixation device being delivered to a targeted facet joint using the delivery instrument in accordance with embodiments of the present disclosure.

FIG. 1E is a side view of the spinal fixation device after implantation in the targeted facet joint in accordance with embodiments of the present disclosure.

FIG. 1F is a side view of four of the spinal fixation devices implanted in consecutive facet joints, along with a connecting rod for coupling the devices in accordance with embodiments of the present disclosure.

FIG. 1G is a perspective view of the vertebrae depicted in FIGS. 1D-1F after placement of four additional spinal fixation devices and two connecting rods in accordance with embodiments of the present disclosure.

FIG. 2A is a perspective view of another spinal fixation device in accordance with embodiments of the present disclosure.

FIG. 2B is an exploded view of the spinal fixation device shown in FIG. 2A.

FIG. 3A is a perspective view of a pre-assembly and post-assembly configuration of a spinal fixation device featuring a tulip screw in accordance with embodiments of the present disclosure.

FIG. 3B is a perspective view of a pre-assembly and post-assembly configuration of another embodiment of the spinal fixation device shown in FIG. 3A in accordance with embodiments of the present disclosure.

FIG. 3C is a perspective view of a pre-assembly and post-assembly configuration of another embodiment of the spinal fixation device shown in FIG. 3A in accordance with embodiments of the present disclosure.

FIG. 3D is a perspective view of a delivery instrument coupled to an embodiment of the cage member shown in FIGS. 3A-3C in accordance with embodiments of the present disclosure.

FIG. 3E is a side view of the cage member of FIG. 3D being delivered to a targeted articular process using the delivery instrument in accordance with embodiments of the present disclosure.

FIG. 3F is a side view of a drill bit creating a pilot hole through the articular process shown in FIG. 3E after placement of the cage member in accordance with embodiments of the present disclosure.

FIG. 3G is a side view of the cage member after placement at the target site in accordance with embodiments of the present disclosure.

FIG. 3H is a side view of the tulip screw coupled with the cage member at the target site depicted in FIG. 3G in accordance with embodiments of the present disclosure.

FIG. 3I is a perspective view of six of the spinal fixation devices shown in FIGS. 3A and 3H placed at consecutive articular processes in accordance with embodiments of the present disclosure.

FIG. 3J is a side view of FIG. 3I, showing a connecting rod coupled with three of the spinal fixation devices in accordance with embodiments of the present disclosure.

FIG. 3K is a perspective view of the vertebrae depicted in FIGS. 3E-3J after placement of six spinal fixation devices and two connecting rods in accordance with embodiments of the present disclosure.

FIG. 4A is a perspective view of a pre-assembly and post-assembly configuration of a spinal fixation device featuring a tulip screw in accordance with embodiments of the present disclosure.

FIG. 4B is a perspective view of a delivery instrument coupled to the cage member of the spinal fixation device of FIG. 4A in accordance with embodiments of the present disclosure.

FIG. 4C is a stepwise representation of a drill bit being inserted through a guide hole defined by a distal end of the delivery instrument shown in FIG. 4B in accordance with embodiments of the present disclosure.

FIG. 4D is a perspective view of the cage member separate from the delivery instrument shown in FIG. 4B in accordance with embodiments of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

As used herein, “vertebrae” may refer to cervical vertebrae unless otherwise indicated. Embodiments are not limited to cervical vertebrae, however, and may encompass other vertebrae or groups of vertebrae along the spine of a subject.

As used herein, “subject” may refer to a human (e.g., patient) afflicted with a spinal condition, e.g., spinal stenosis, injury or disease, which may be degenerative. The condition may impact the integrity and/or positioning of one or more vertebrae, which may cause chronic pain and instability.

As used herein, “device” may refer to a spinal fixation device, rod fixation device, or component(s) thereof. At least a portion of the device, e.g., the distal end, may be inserted into a cervical facet joint. Accordingly, the disclosed devices, components and/or portions thereof may, in some examples, be considered or referred to as intrafacet implants. At least another portion of the device, e.g., the tulip member, may be configured to couple and secure a connecting rod.

The terms “distal” and “proximal” are used to refer to a position or direction relative to the treating clinician, such as a surgeon. “Distal” and “distally” refer to a position that is distant from, or in a direction away from, the treating clinician. “Proximal” and “proximally” refer to a position that is near, or in a direction toward, the treating clinician.

For the following defined terms, certain definitions shall be applied unless a different definition is given elsewhere in this disclosure. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.).

Surgical solutions to spinal conditions often require the use of a variety of hardware components, such as rods and bone screws, to correct and provide rigidity to and between vertebrae. The screws are often inserted into adjacent vertebrae (e.g., consecutive spine segments such as C5 and C6) and then connected via rods to prevent motion at the connected segments being fused. The screws may thus provide anchors for the rods and additional stability to the fusion site.

While systems featuring a combination of connecting rods and bone screws may provide stability to the vertebrae, fastening the rods using screws inserted directly into the bones is an invasive and risky process requiring dangerous drilling that must be performed with high precision (with respect to location, trajectory and depth) near critical anatomy (e.g., vasculature, nerves, and the spinal cord) to ensure each screw is placed at the correct trajectory within a vertebrae structure. This procedure is further complicated by the close proximity of other anatomical features and substances (e.g., blood, ligaments, muscles, etc.), which may mask or interfere with the physical interactions of the instruments designed to place the screw accurately.

Some spinal fixation systems may require “tulip” members for securing bone screws to the vertebrae and supporting connecting rods laterally along or across the vertebrae. A single tulip member may be configured to couple with a complementary screw drilled directly into the bone and support a connecting rod placed over the proximal end of the screw. In addition or alternatively, certain spinal fixation systems may include facet joint implants placed in the facet joints defined by adjacent articular processes. Such implants may be inserted independently from the connecting rods and bone screws, where the implants may fuse and support adjacent vertebrae by providing a rigid connection point for the connecting rods to traverse multiple spinal levels.

Embodiments disclosed herein include spinal fixation devices, systems and components thereof, along with associated methods of assembly and use. Examples of a disclosed device include a singular construct comprising a cage member and an articulating tulip member, the cage member configured for implantation in a facet joint and the tulip member movably coupled to the proximal end of the cage member via one or more connecting members or linkages. After implantation in a facet joint, the cage member may securely anchor one or more connecting rods via the tulip member, which may rotate and pivot relative to the cage member about a ball joint and pivot joint, respectively. The ball and/or pivot joints included in embodiments of the disclosed device provide additional degrees of movement for the tulip relative to preexisting rods and screws. By being configured to insert in a facet joint and secure vertebrae connecting rods, some embodiments of the devices disclosed herein may eliminate or at least reduce the need for drilling bone screws directly into the vertebrae. The devices may thus enable a more safe and consistent approach to spinal fixation.

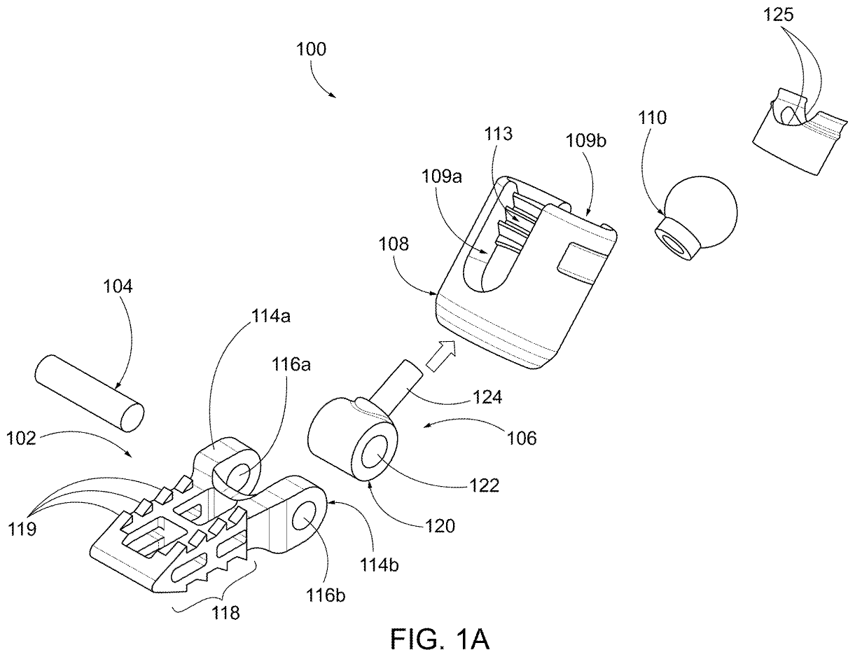

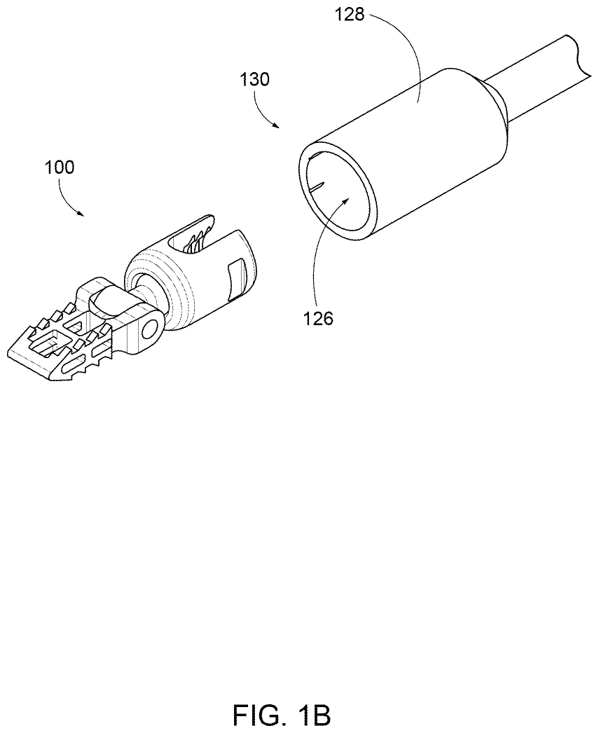

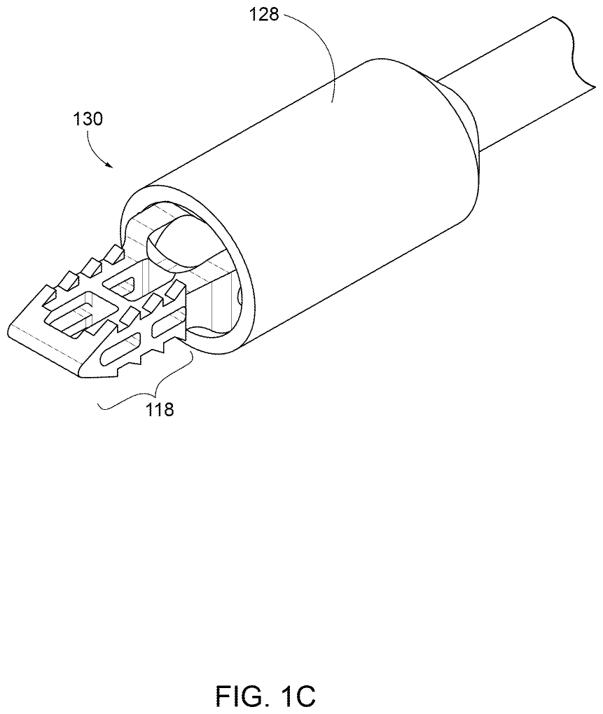

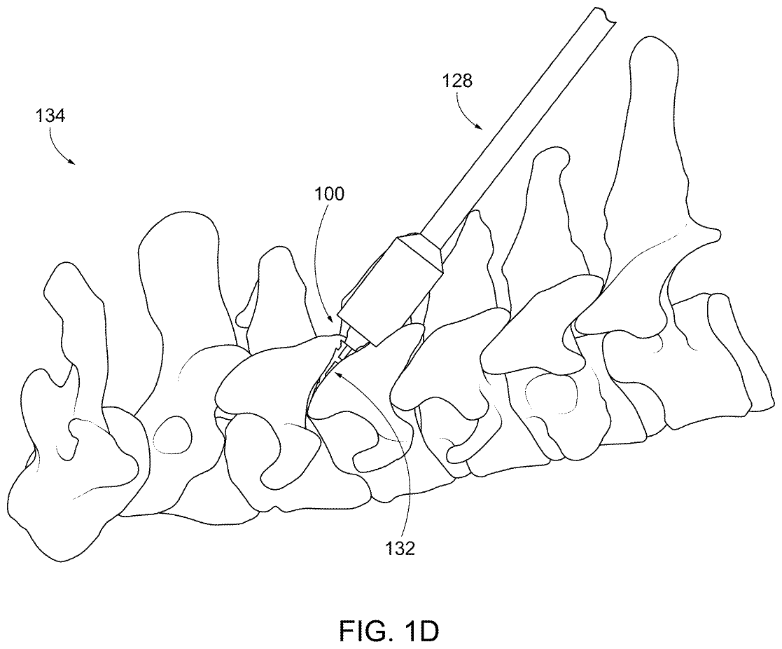

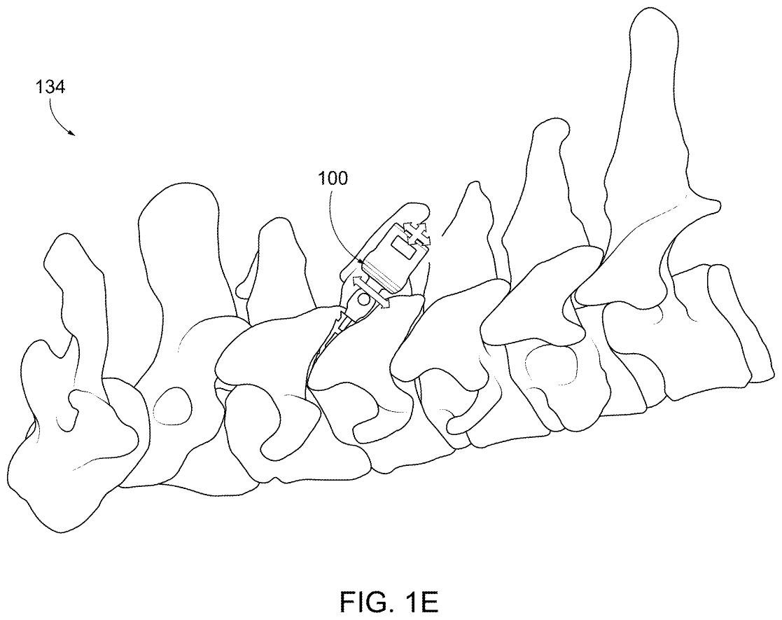

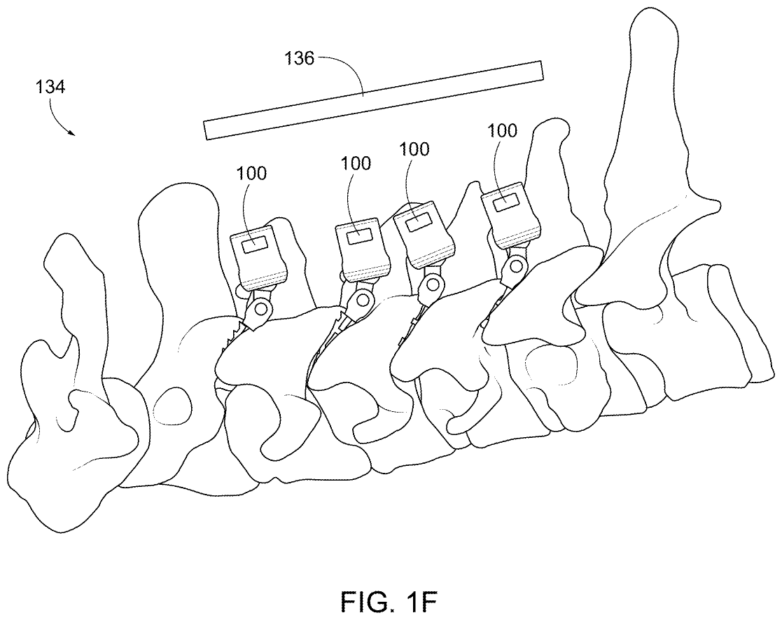

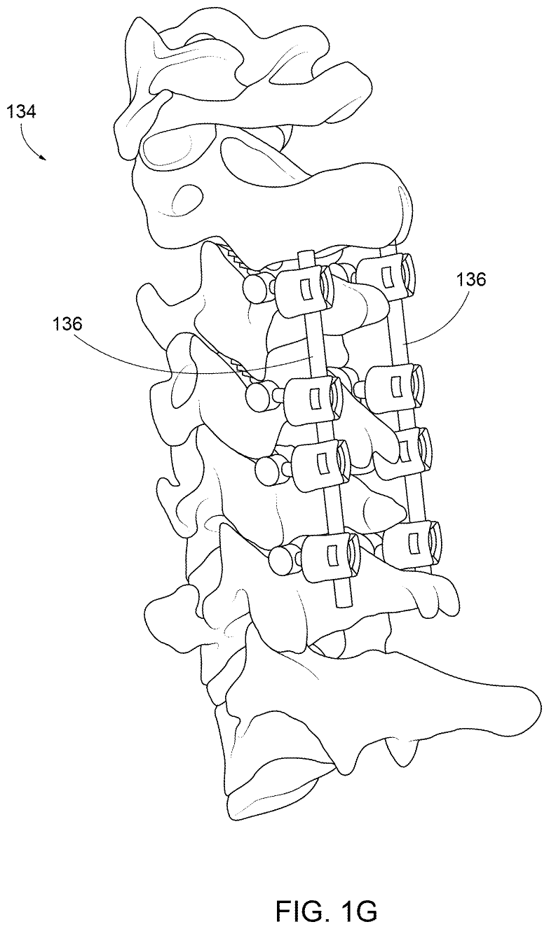

FIG. 1A shows an exploded view of an embodiment of a device 100 for spinal fixation. As shown, the device 100 includes a cage member 102, a pivot pin 104, a pivot member 106, a tulip member 108, a tulip ball 110, and a tulip cap 112. For ease of illustration, the tulip ball 110 may be considered or referred to as the ball joint herein, and the combination of the pivot pin 104 and pivot member 106 likewise referred to as the pivot joint. Although reference is made to a tulip with respect to tulip member 108, tulip ball 110, and tulip cap 112, “tulip” is used as one example for convenience and without intent to limit. For example, embodiments may include any articulating device or assembly that receives a rod, as described herein. Thus, tulip member 108 may be referred to as an articulating member, tulip ball 110 may be a ball joint, and tulip cap 112 may simply be a cap.

The cage member 102 may include proximal arm or shoulder components 114a, 114b each defining a through-hole 116a, 116b sized and configured to receive the pivot pin 104. A portion of the cage member 102, e.g., at least a distal end portion, may include or define an intrafacet insert portion 118 configured to be placed or implanted in a facet joint between adjacent vertebrae. The intrafacet insert portion 118 may have a textured exterior surface and/or may include distinct teeth, raised ridges, serrations, or other protruding features 119 configured to engage a facet joint. The configuration of the intrafacet insert portion 118 may include a variety of additional or alternative features, including distally tapered, beveled or chamfered surfaces and/or expandable components, the latter of which may include arms held in a closed position by a detent feature.

The pivot member 106 may include a cylindrical body 120 sized and configured to fit between the two shoulder portions 114a, 114b of the cage body 102 and defining an inner lumen or through-hole 122 sized and configured to the receive pivot pin 104. A shaft portion 124 extends from an outer surface of the cylindrical portion 120. As shown in this example, the shaft portion 124 may have a smooth or substantially smooth surface and may be arranged substantially perpendicular to the longitudinal axis of the cylindrical portion 120 and through-hole 122 defined therein.

The shaft portion 124 may be inserted through an opening at the bottom or distal end of the tulip member 108, in the direction indicated by the arrow. Once inserted into the cavity defined by the tulip member 108, the proximal end of the shaft portion 124 may be covered by the tulip ball 110. The tulip cap 112 may then be inserted within the cavity defined by the tulip until the cap contacts or couples with the tulip ball 110, such that the concave surfaces 125 defining the proximal end of the tulip cap 112 are positioned opposite the ball 110.

As further shown, the tulip member 108 may define two opposing slots 109a, 109b configured to receive and support a connecting rod, which may be slid down (distally) or otherwise inserted in the slots 109a, 109b until seated on the complementary concave surfaces of the tulip cap 112, which may align with the concave surfaces defined by the bottom (or distal end) of each slot 109a, 109b.

The combination of the pivot joint 106 and ball joint 110 enables the tulip member 108 to move or articulate in multiple directions after implantation at a facet joint. For example, the tulip member 108 may be coupled to the cage member 102 via one or more pivot joints, such as via an articulating joint (e.g., ball joint 110) and a pivot joint (e.g., pivot joint 106). The tulip member 108 may move bi-directionally about the pivot joint 106 and rotate freely about the ball joint 110. Movement of the tulip member 108 in this manner may enable a clinician to adjust its position and/or orientation after device placement in the manner necessary to accommodate various positions and orientations of the targeted vertebrae and the connecting rods used to secure the same. This versatility of the device 100 may reduce the complexity, time and difficulty of spinal fixation procedures, all without the use of invasive bone screws.

Such embodiments are illustrative only, and the tulip member 108 may be movably coupled to the cage member 102 in other configurations. For example, pivot member 106 and tulip member 108 may be fixed to each other (e.g., as a single piece), such that the tulip member 108 pivots about pivot pin 104 without articulation relative to pivot member 106 (e.g., about ball joint 110). In such embodiments, the pivot member 106 may slide along the pivot joint, such as sliding laterally along pivot pin 104 to provide lateral adjustability. Alternatively, pivot member 106 may be fixed to cage member 102 (e.g., at a fixed angle relative to cage member 102), leaving the articulating ball joint 110 to address the needed adjustability of the device.

As further shown, the tulip member 108 may define a threaded internal surface 113 configured to receive additional surgical instruments, such as a set screw, which may be inserted on a connecting rod resting in the tulip member 108, thereby locking the rod in place.

A press fit assembly method may be used to assemble the device 100, which may be made of a variety of materials, non-limiting examples of which may include titanium, stainless steel, spring steel, nitinol, polycarbonate, or any other metal, metal alloy or polymer of sufficient strength. Different features or components of the device may be made of different materials.

As shown in FIGS. 1B and 1C, the assembled device 100 may be at least partially inserted, fastened or otherwise coupled within an inner lumen 126 of an elongate delivery instrument 128, such that the device is kept aligned and rigid before and during implantation in a facet joint. As shown particularly in FIG. 1C, the device 100 may be inserted into the distal end 130 of the delivery instrument 128 until only the intrafacet insertion portion 118, or a portion thereof, remains at least partially protruding from the distal end 130, thereby enabling insertion of the device 100 at the target site. Together, the device 100 and delivery instrument 128 may constitute at least a portion of a spinal fixation access and delivery system.

FIG. 1D illustrates a snapshot of a spinal fixation process performed using the device 100. As shown, the device 100 and delivery instrument 128, once coupled, may be inserted by a surgeon through an incision formed at the surgical site and the distal portion 130 of the delivery instrument 128 advanced to a targeted facet joint 132 nestled within the cervical vertebrae 134 of a subject. After the device 100 is successfully placed at the facet joint 132, the delivery instrument 128 may be detached from the device 100 and removed from the subject, as shown in FIG. 1E. Unconstrained from the inner lumen 126 of the delivery instrument 128, the device 100 may now be rotated freely about the ball joint and pivoted about the pivot joint, as indicated by the arrows.

The device implantation process can be repeated as many times as necessary for a given procedure, for example at least until two or more devices 100 are implanted into respective facet joints (FIG. 1F). A connecting rod 136 may be coupled across the tulip members of the implanted devices 100. As evident in FIG. 1F, accommodating the connecting rod 136 across uneven and/or misaligned vertebrae may require the tulip members 108 to move independently about their respective pivot and/or ball joints until reaching the necessary positions and orientations. Articulation of the tulip members in this manner may reduce or eliminate the need to place each device at a specific position and/or angle at the outset, as would be required using preexisting devices lacking the ball and/or pivot joints in a singular device construct and thus not easily adjustable after initial device placement. Multiple sets of devices 100 may be utilized to secure separate rods 130 and form a complete spinal fixation construct, as shown in FIG. 1G.

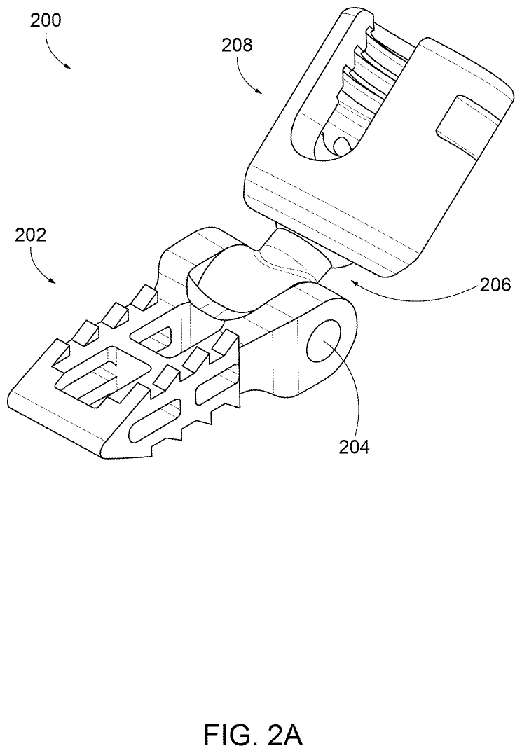

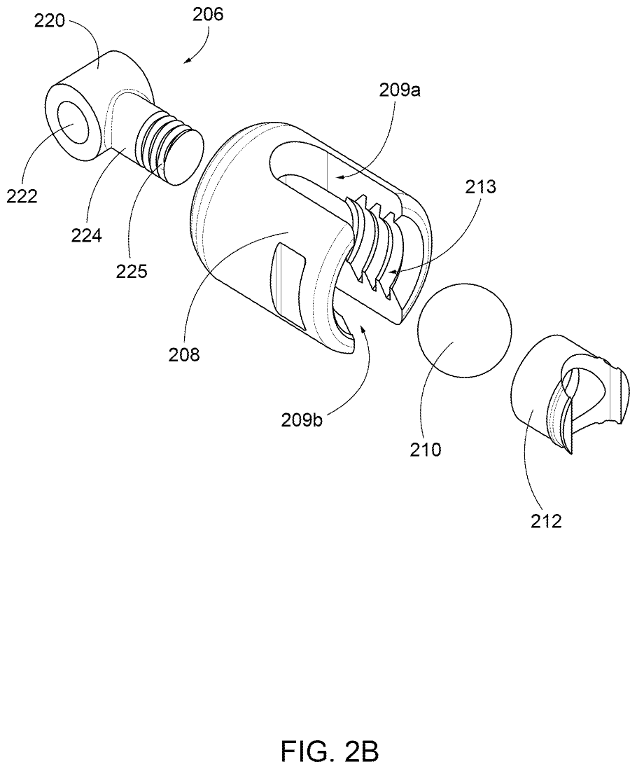

FIGS. 2A and 2B depict another device 200 configured for securing connecting rods across one or more vertebrae of a subject. Like device 100, the device 200 includes a cage member 202, a pivot pin 204, a pivot member 206, a tulip member 208 defining opposing slots 209a, 209b and a threaded interior surface 213, a tulip ball 210, and a tulip cap 212. The pivot member 206 includes a cylindrical body 220 and a through-hole 222, but unlike device 100, the shaft portion 224 of the pivot member 206 defines a threaded exterior surface 225. To engage the threaded exterior surface 225 of the shaft portion 224 and secure the tulip member 208 to the cage member 202, the tulip ball 210 may define a complementary threaded interior surface. This configuration allows the device 200 to be assembled via a screw assembly method which involves, among other steps, inserting a proximal end of the shaft portion 224 through a hole defined by the bottom (distal) end of the tulip member 208 and screwing the tulip ball 210 onto the threaded exterior surface 225 of the shaft portion 224 until the two components are securely fastened. The device 200 may otherwise function in the same manner as device 100, such that the tulip member 208 may be configured to move about the pivot joint and ball joint after being placed at a facet joint and released from a delivery instrument.

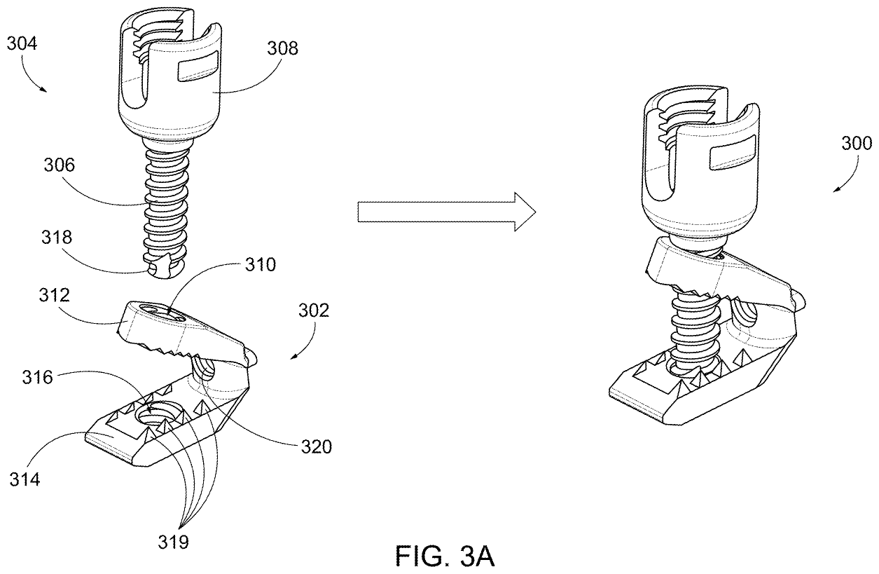

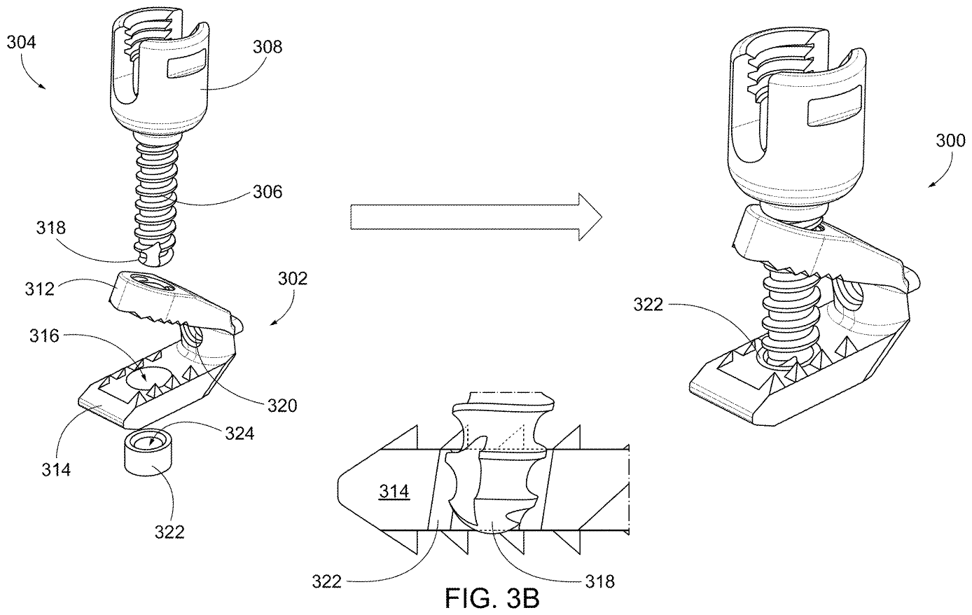

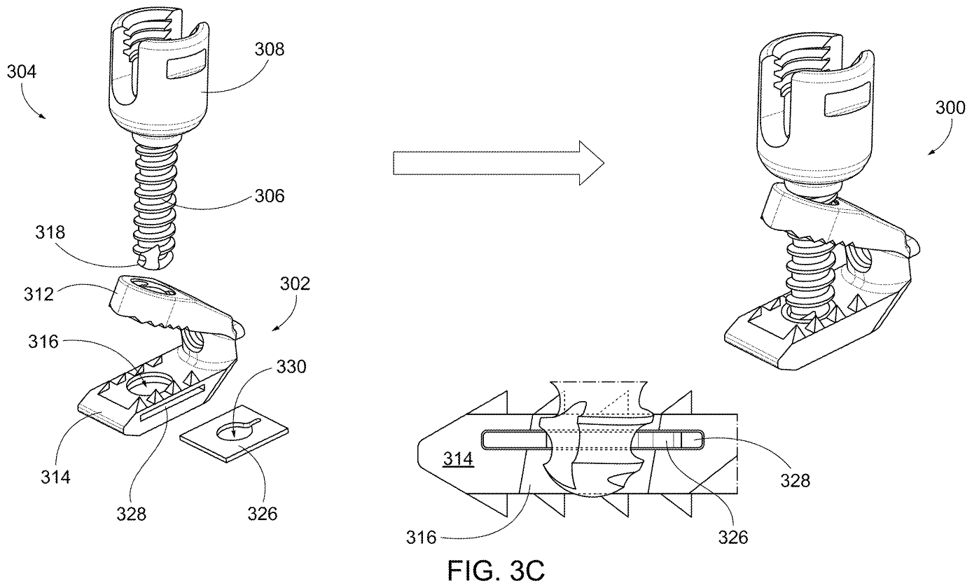

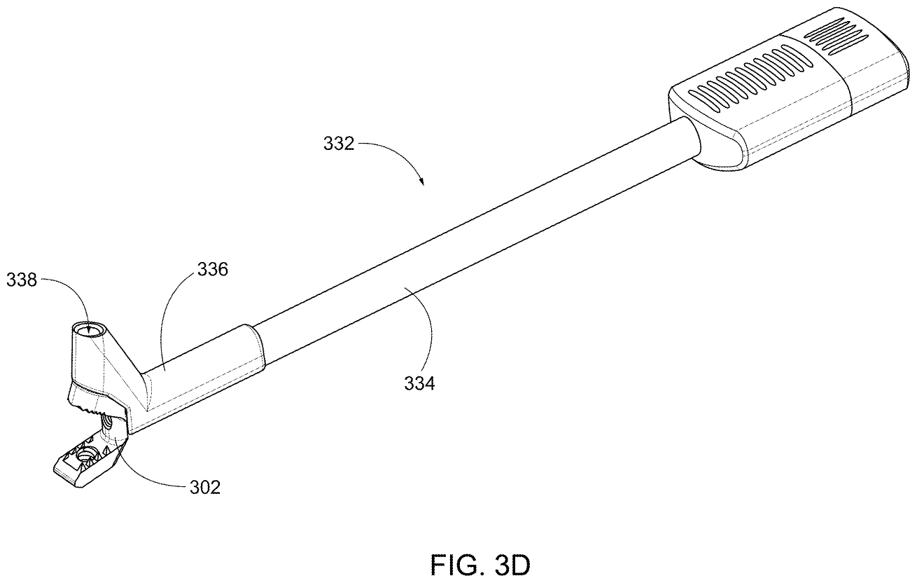

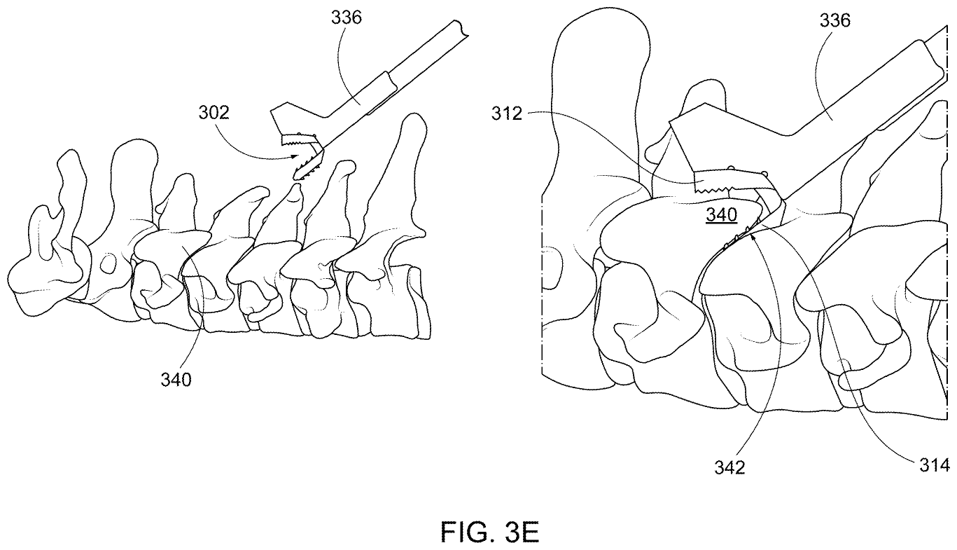

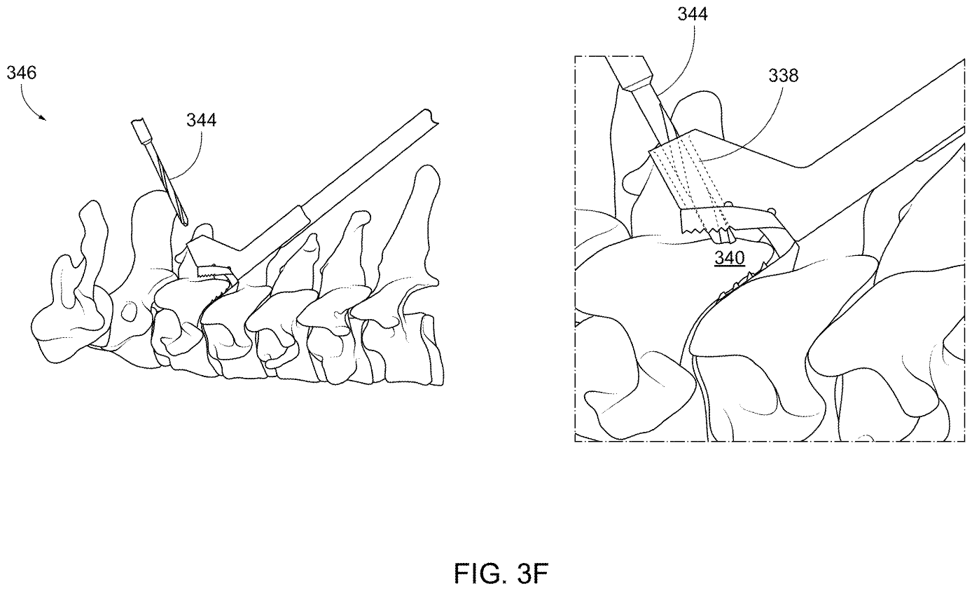

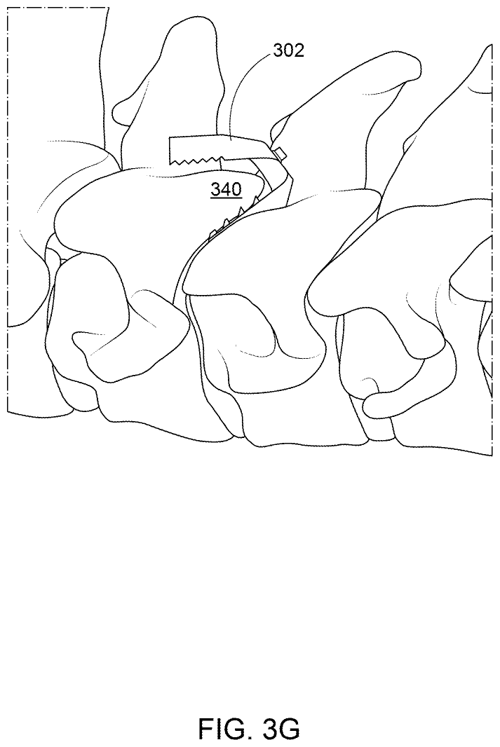

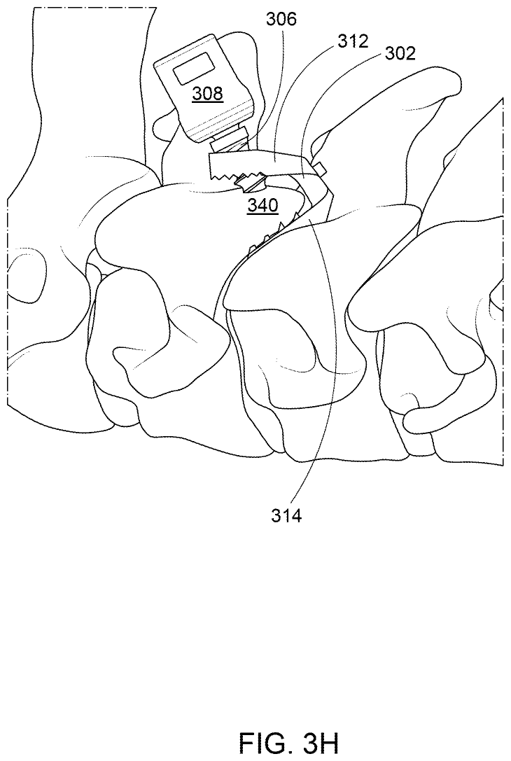

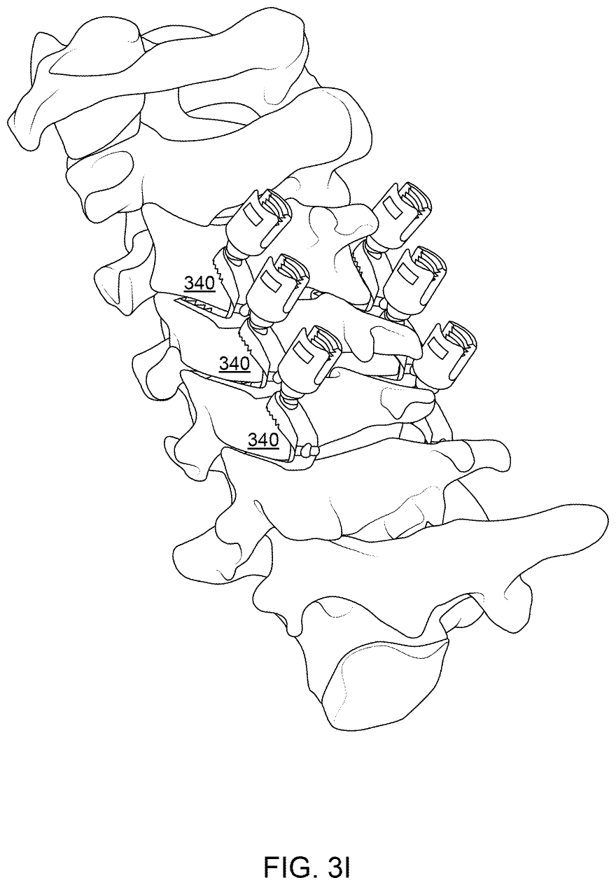

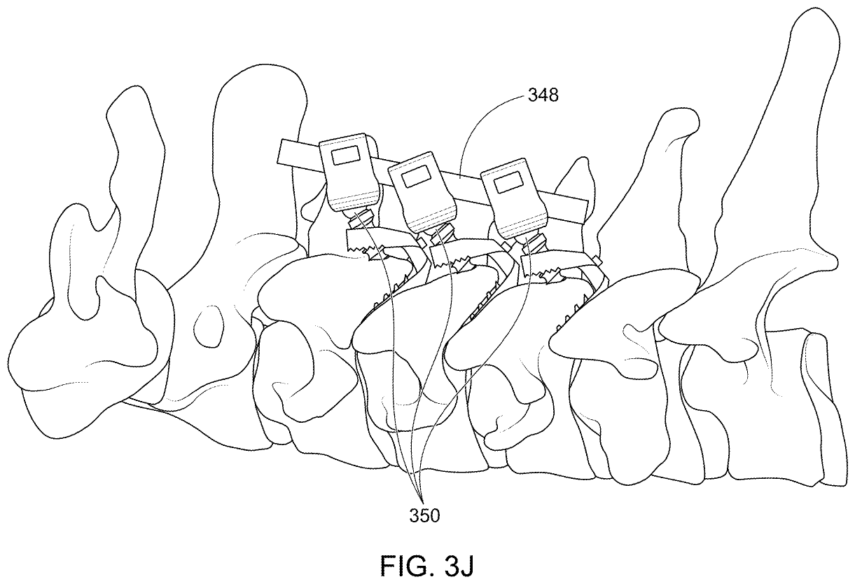

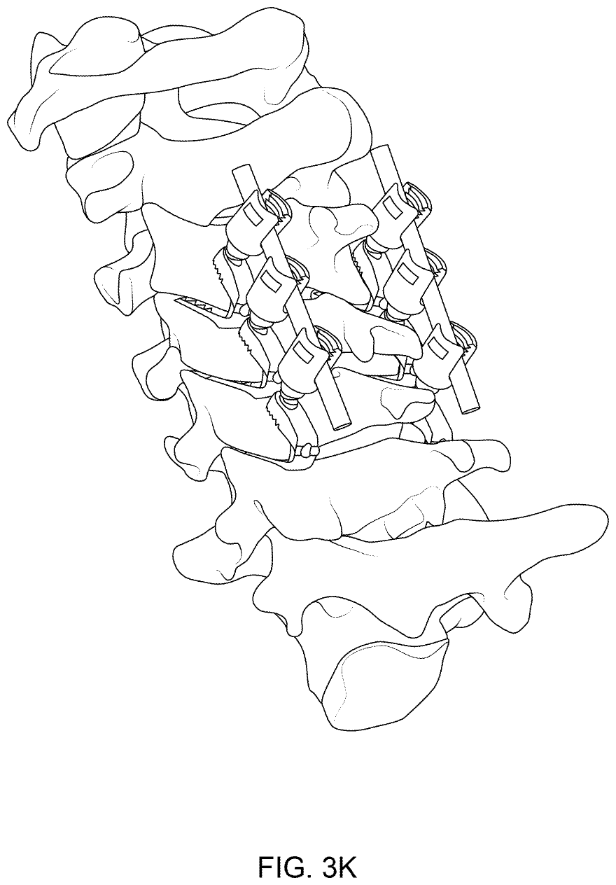

FIG. 3A shows a device 300 featuring a cage member 302 and tulip screw 304. As shown, the tulip screw 304 may include an elongate screw portion 306 extending from the body of a tulip member 308. A tulip ball may be secured at a proximal end of the screw portion 306 within the cavity defined by the tulip member 308 to enable movement of the tulip member 308 after insertion at a target site. The elongate screw portion 306 is configured for insertion through a first hole 310 defined by a first arm member 312 of the cage member 302. A second arm member 314 of the cage member 302 includes a second hole 316, which may comprise a through-hole or blind hole configured to receive and prevent further distal movement of a distal end 318 of the screw portion 306, such that the distal end 318 of the screw portion 306 is firmly seated in the second hole 316. The screw portion 306 features a threaded exterior surface configured to engage the threaded interior surfaces of both the first hole 310 and the second hole 316. As further illustrated, one or both arm members 312, 314 may have one or more textured surfaces and/or may include distinct teeth, raised ridges, serrations, or other protruding features 319 configured to engage an articular process, thereby positioning the second arm member 314 within a facet joint. The device 300 also includes a delivery instrument coupling hole 320 having a threaded interior surface, configured to engage a complementary threaded pin or screw portion defined by a distal end of a delivery instrument.

As shown in FIG. 3B, the second hole 316 defined by the second arm member 314 may comprise a through-hole having a substantially smooth, not threaded, interior surface. Such embodiments may further include an insert 322, e.g., a nylon insert, configured to be inserted within the second hole 316. The insert 322 may define a through-hole, blind hole or cavity 324 configured to receive and prevent further distal movement of the distal end 318 of the screw portion 306, as shown in the cross-sectional view of FIG. 3B. The insert 322 may be made of a variety of materials, e.g., polymers, and is not limited to nylon.

As shown in FIG. 3C, another embodiment of the device 300 may include or be coupled with a lock shim insert 326 configured to be inserted into a side slot 328 defined by the second arm member 314 and advanced laterally until the aperture 330 of the insert is concentric with the second hole 316. The distal end 318 of the screw portion 306 may be inserted through the aperture 330 and secured or locked in place by constriction of the shim insert 326.

As shown in FIG. 3D, the cage member 302 may be coupled with an elongate delivery device 332 configured to place the cage member at a target site, such as an articular process, where the arm members 312, 314 may straddle the process. The elongate delivery instrument 332 may include an elongate shaft member or shaft portion 334 coupled at a distal end with a cage adapter 336 that couples with the proximal or back end of the cage member 302 and defines a drill bit guide hole 338 configured to receive and guide a drill bit through the first and second holes of the cage member 302. During a spinal fixation procedure, the drill bit may be used to drill a pilot hole through an articular process positioned between the arm members 312, 314 after placement of the cage member 302 at a target site.

In operation, the cage member 302 and delivery instrument 332 coupled thereto may be inserted by a surgeon through an incision formed at the surgical site and the cage member 302 advanced toward a targeted articular process 340 until the arm members 312, 314 of the cage member straddle opposing surfaces of the process 340 and the second arm 314 is positioned in an adjacent facet joint 342 (FIG. 3E). With the delivery instrument 332 holding the cage member 302 in position, a drill bit 344 coupled to a drilling device 346 may be inserted through the drill bit guide hole 338 and extended through the first hole 310 of the first arm member 312, after which the drill bit 344 may be drilled through the articular process 340, forming a pilot hole for receipt of the screw portion 306 of the tulip screw 304 (FIG. 3F). The drill bit 344 may be removed when drilling is complete, along with the delivery instrument 332, leaving the cage member 302 in place (FIG. 3G). The tulip screw 304 may then be inserted and tightened into the cage member 302 by inserting the elongate screw portion 306 through the first hole 310 of the cage member 302, the pilot hole drilled through the articular process 340, and subsequently into the second hole 316 of the second arm member 314, where the distal end 318 is firmly seated (FIG. 3H). This process may be repeated as many times as necessary, until two or more devices 300 are secured to respective articular processes 340 (FIG. 3I). A connecting rod 348 may be subsequently coupled across the tulip members 308 of the devices 300, and as illustrated in FIG. 3J, accommodating the connecting rod 348 across uneven and/or misaligned vertebrae may require the tulip members 308 to move independently about their respective ball joints 350 until reaching the necessary position and orientation. Multiple sets of devices 300 may be utilized to secure separate rods 348 and form a complete spinal fixation construct, as shown in FIG. 3K.

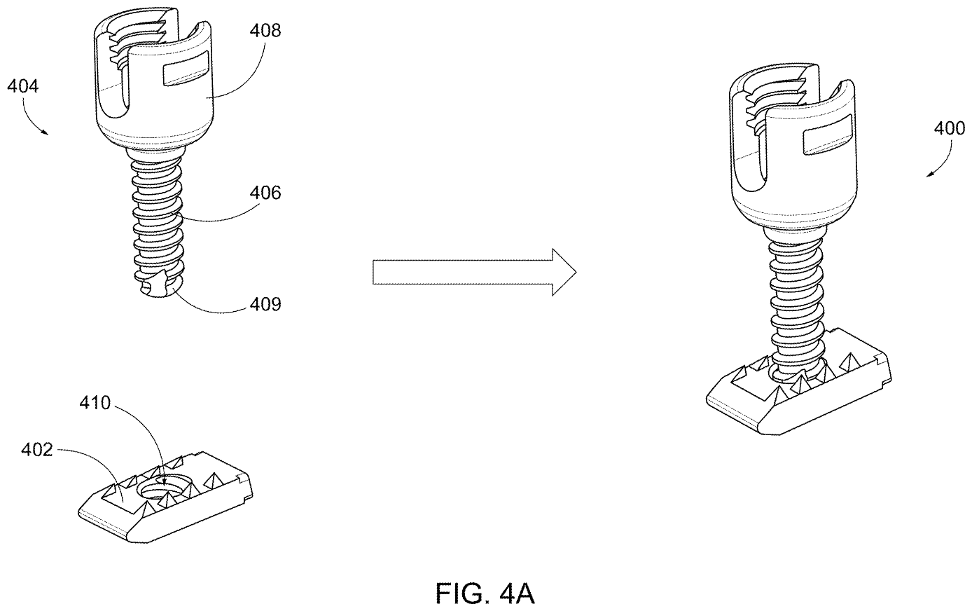

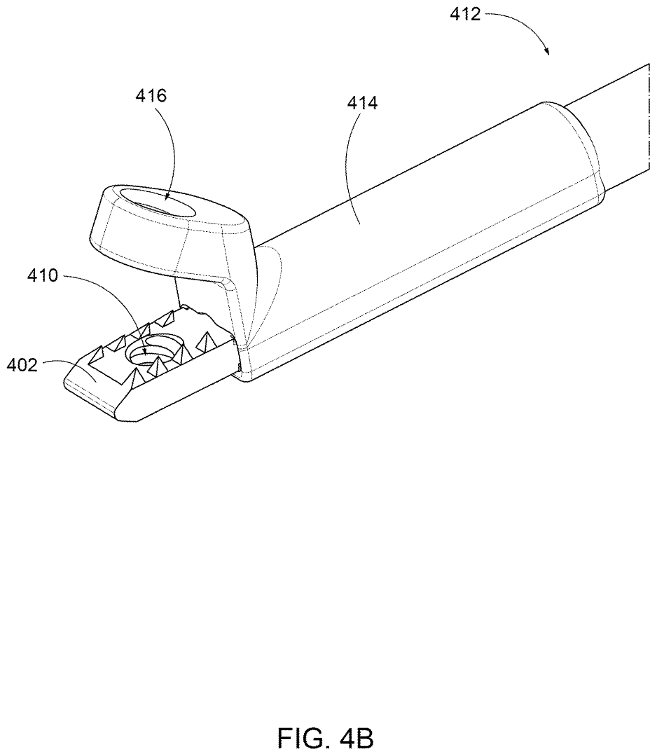

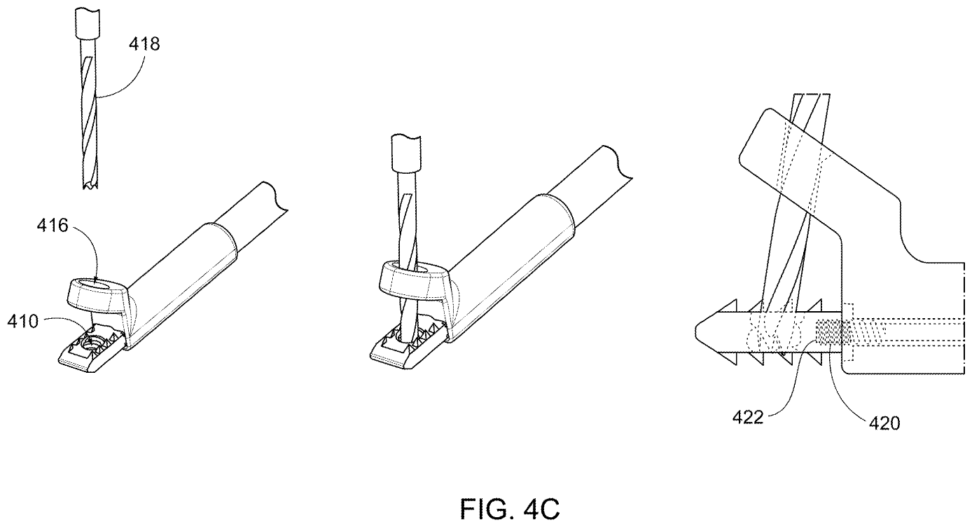

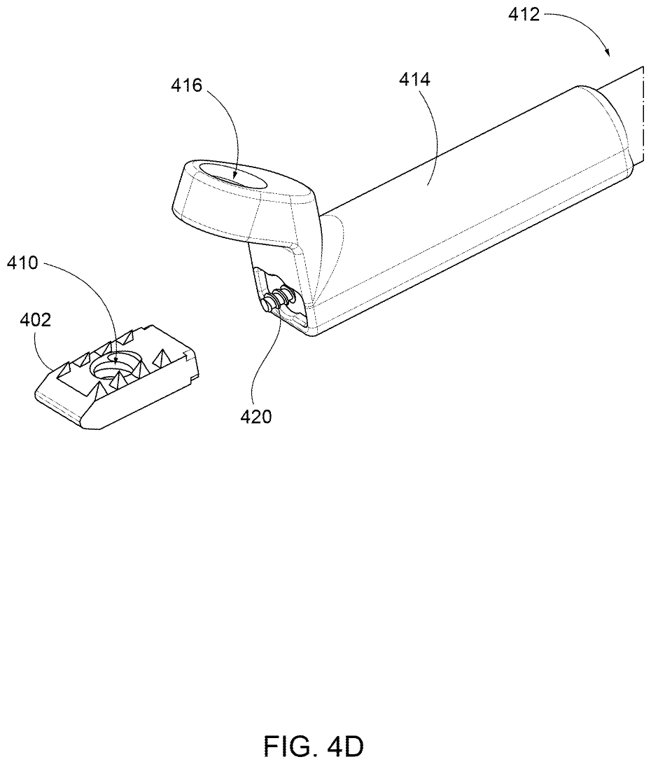

FIG. 4A shows another device 400 comprising a cage member 402 and a complementary tulip screw 404, the latter featuring an elongate screw portion 406 extending from a tulip member 408. As shown, the cage member 402 may comprise a unitary body that defines a tulip screw receiving hole 410 and having a threaded interior surface configured to engage the complementary exterior threaded surface of the elongate screw portion 406, such that a distal end 409 of the screw portion 406 can be firmly seated in the receiving hole or opening 410.

As shown in FIG. 4B, the cage member 402 may be coupled to a distal end of an elongate delivery instrument 412 configured to place the cage member at a target site, such as an articular process, where the cage member 402 may be inserted in a facet joint. The elongate delivery instrument 412 may include or be coupled with a cage adapter 414 that couples to the proximal or back surface of the cage member 402 and defines a drill bit guide hole 416 configured to receive and guide a drill bit through an articular process and into the receiving hole 410 of the cage member 402. FIG. 4C illustrates the insertion of the drill bit 418 through the drill bit guide hole 416 and into the tulip screw receiving hole 410. The figure also provides a view of a distal attachment screw 420 protruding from the distal end of the cage adapter 414. The attachment screw 420 defines a threaded exterior surface configured to engage a complementary threaded interior surface defined by a screw receiving hole or opening 422 extending into the back end of the cage member 402.

FIG. 4D shows the cage member 402 detached from the delivery instrument adapter 414, revealing the distal attachment screw 420 protruding from the distal end of the delivery instrument. The attachment screw 420 may be received within the complementary screw receiving hole 422 defined within the cage member 402, perpendicular to the longitudinal axis of the receiving hole 410. Additional coupling mechanisms may be included in lieu of or in addition to the mechanism shown in FIG. 4C. A snap-fit or lock-and-key mechanism, for instance, may be utilized to couple the cage member to the delivery instrument.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A device for spinal fixation, the device comprising:
a cage member defining a body configured for insertion into a facet joint; and
an articulating member configured to receive a connecting rod and coupled to a proximal end of the cage member via at least one pivot joint.

2. The device of claim 1, wherein the articulating member defines a connecting rod receiving slot defining a proximal open end and a concave resting surface opposite the proximal open end and complementary to the connecting rod.

3. The device of claim 1, wherein the at least one pivot joint comprises a pivot pin and a pivot member, the pivot member defining an inner lumen configured to receive the pivot pin.

4. The device of claim 3, wherein the proximal end of the cage member defines opposing shoulder portions each defining through-holes configured to receive the pivot pin and align concentrically with the inner lumen of the pivot member.

5. The device of claim 3, further comprising a ball joint coupled to the at least one pivot joint and positioned at least partially within a cavity defined by the articulating member, wherein the ball joint comprises a ball member coupled to a shaft member, the shaft member extending from the pivot member.

6. The device of claim 5, wherein the shaft member is arranged substantially perpendicular to the inner lumen of the pivot member.

7. The device of claim 6, wherein an exterior surface of the shaft member is selected from threaded and substantially smooth.

8. (canceled)

9. The device of claim 1, wherein the articulating member is configured to pivot about and/or slide along the pivot joint.

10. The device of claim 1, wherein the body of the cage member comprises protrusions configured to engage the facet joint.

11. The device of claim 1, further comprising a ball joint coupled to the at least one pivot joint and positioned at least partially within a cavity defined by the articulating member, such that the articulating member is configured to rotate about the ball joint.

12. The device of claim 11, further comprising a cap coupled to the ball joint within the cavity defined by the articulating member and defining concave surfaces complementary to the connecting rod.

13. The device of claim 1, wherein the at least one pivot joint comprises an articulating joint and a pivot joint.

14. A device for spinal fixation, the device comprising:
a cage member defining a body comprising two arm members configured to engage an articular process therebetween;
an articulating member configured to receive a connecting rod and defining an elongate screw portion, the elongate screw portion defining a threaded exterior surface configured to extend through and engage complementary holes defined by the arm members; and
a ball joint coupled to a proximal end of the elongate screw member and positioned at least partially within a cavity defined by the articulating member.

15. The device of claim 14, wherein one of the two arm members is configured for insertion into a facet joint.

16. The device of claim 14, wherein each of the complementary holes defined by the arm members defines a threaded interior surface complementary to the threaded exterior surface of the elongate screw portion.

17. The device of claim 14, further comprising a nylon insert configured to be inserted into one of the holes defined by the arm members.

18. The device of claim 14, further comprising a lock shim insert configured to be inserted into a complementary side slot defined by one of the two arm members.

19. (canceled)

20. The device of claim 14, further comprising a coupling mechanism for coupling the device with a delivery instrument, wherein the coupling mechanism comprises a threaded receiving hole configured to receive a threaded screw portion extending from a distal end of the delivery instrument.

21. (canceled)

22. The device of claim 14, wherein the articulating member is configured to rotate about the ball joint.

23. The device of claim 14, wherein the articulating member defines a connecting rod receiving slot defining a proximal open end and a concave resting surface opposite the proximal open end and complementary to the connecting rod.

24.-27. (canceled)
